(19) United States
(12) Patent Application Publication
(10) Pub. No.: US 2021/0310708 A1
(43) Pub. Date: Oct. 7, 2021

(54) Refrigerant Identification Assembly

(71) Applicant: Philip Brash, Fredericksburg, VA (US)

(72) Inventor: Philip Brash, Fredericksburg, VA (US)

(21) Appl. No.: 16/837,118

(22) Filed: Apr. 1, 2020

(51) Int. Cl. F25B 49/02; G01N 33/00

(52) U.S. Cl. CPC F25B 49/02; G01N 33/00

(57) ABSTRACT

A refrigerant identification assembly includes a housing that has a high pressure inlet, a low pressure inlet and a purge outlet. A high pressure hose is fluidly coupled to the high pressure inlet for fluidly coupling to a high pressure refrigerant line of an HVAC system. A low pressure hose is fluidly coupled to the low pressure inlet for fluidly coupling to a low pressure refrigerant line of an HVAC system. A refrigerant sensor is positioned within the housing and the refrigerant sensor is in fluid communication with each of the high pressure inlet and the low pressure inlet. In this way the refrigerant sensor can sense the type of refrigerant used in the air conditioning system. A refrigerant display is coupled to the housing and the refrigerant display is in communication with the refrigerant sensor to communicate the type of refrigerant to the user.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to identification devices and more particularly pertains to a new identification device for electronically identifying the type of refrigerant in an HVAC system.

### (2) Description of Related Art Including Information Disclosed Under 37 CFR 1.97 and 1.98

The prior art relates to identification devices which includes a refrigerant service device for removing water and oil from a gaseous refrigerant. The prior art also discloses a refrigerant leak detector for detecting a refrigerant leak in a closed heat exchanging system. The prior art discloses a diagnostic device that measures air temperature and air pressure of an HVAC system for calculating the superheat condition of the HVAC system. The prior art discloses an acid sensor that employs infrared radiation to identify various organic and inorganic acids in a refrigeration unit. The prior art further discloses a diagnostic computer that detects faults and provides diagnostic information for a refrigeration system.

### BRIEF SUMMARY OF THE INVENTION

An embodiment of the disclosure meets the needs presented above by generally comprising a housing that has a high pressure inlet, a low pressure inlet and a purge outlet. A high pressure hose is fluidly coupled to the high pressure inlet for fluidly coupling to a high pressure refrigerant line of an HVAC system. A low pressure hose is fluidly coupled to the low pressure inlet for fluidly coupling to a low pressure refrigerant line of an HVAC system. A refrigerant sensor is positioned within the housing and the refrigerant sensor is in fluid communication with each of the high pressure inlet and the low pressure inlet. In this way the refrigerant sensor can sense the type of refrigerant used in the air conditioning system. A refrigerant display is coupled to the housing and the refrigerant display is in communication with the refrigerant sensor to communicate the type of refrigerant to the user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a refrigerant identification assembly according to an embodiment of the disclosure.

FIG. 2 is a back view of an embodiment of the disclosure.

FIG. 3 is a bottom view of an embodiment of the disclosure.

FIG. 4 is a front view of an embodiment of the disclosure.

FIG. 5 is a left side view of an embodiment of the disclosure.

FIG. 6 is a perspective in-use view of an embodiment of the disclosure.

FIG. 7 is a schematic view of an embodiment of the disclosure.

### DETAILED DESCRIPTION OF THE INVENTION

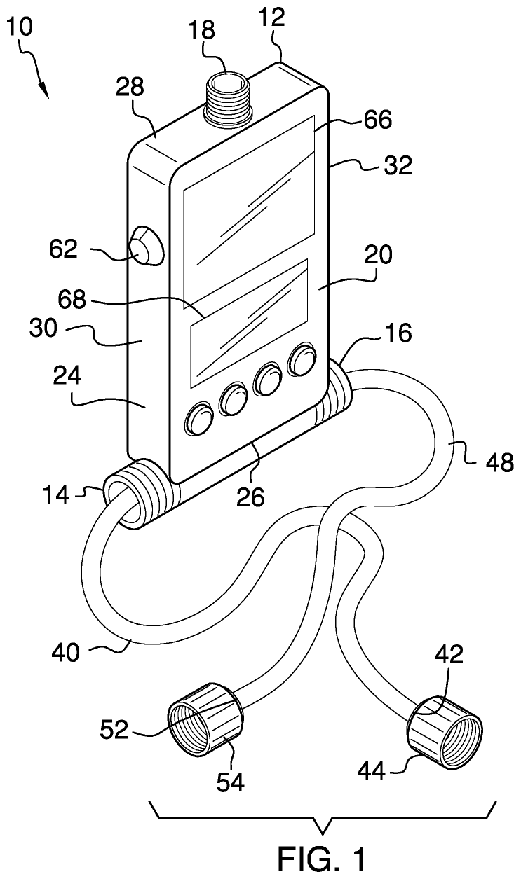

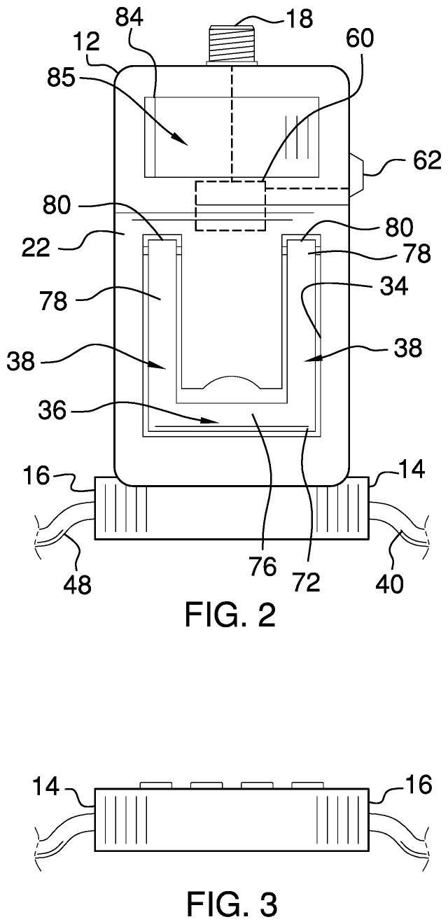

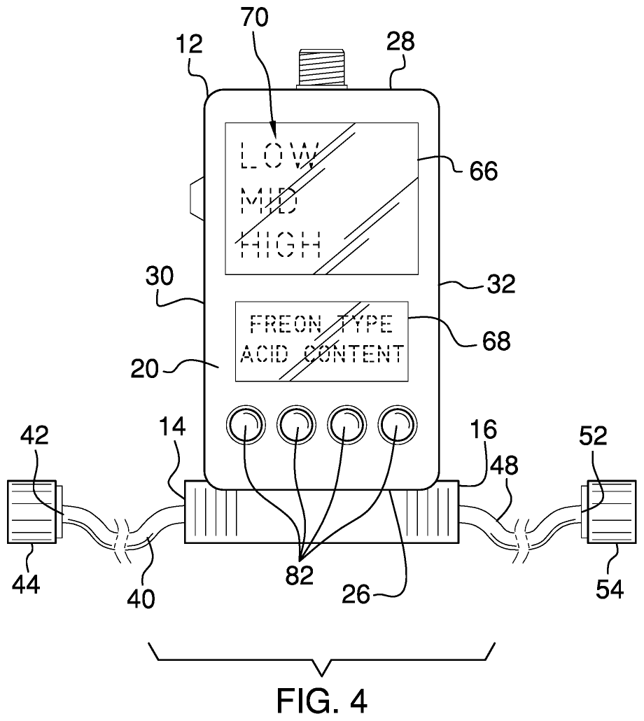

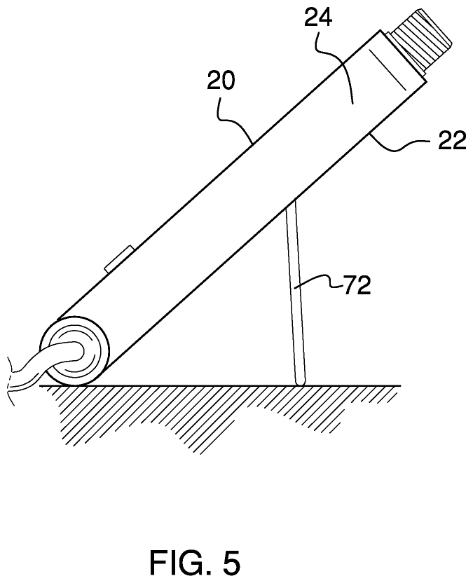

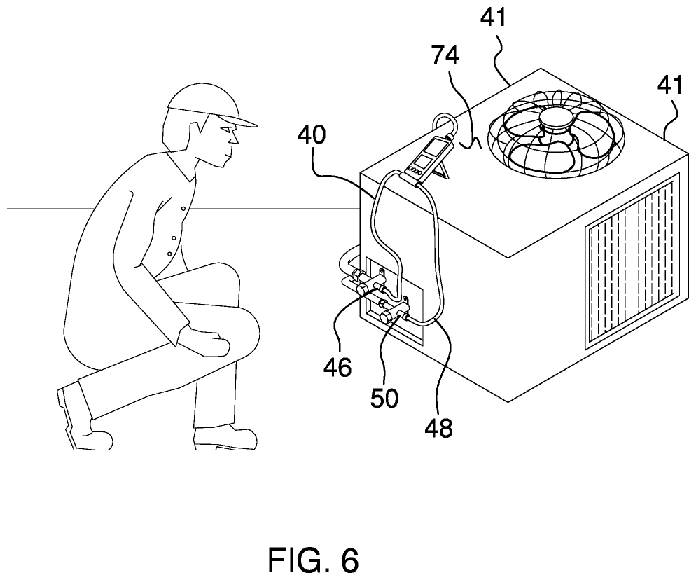

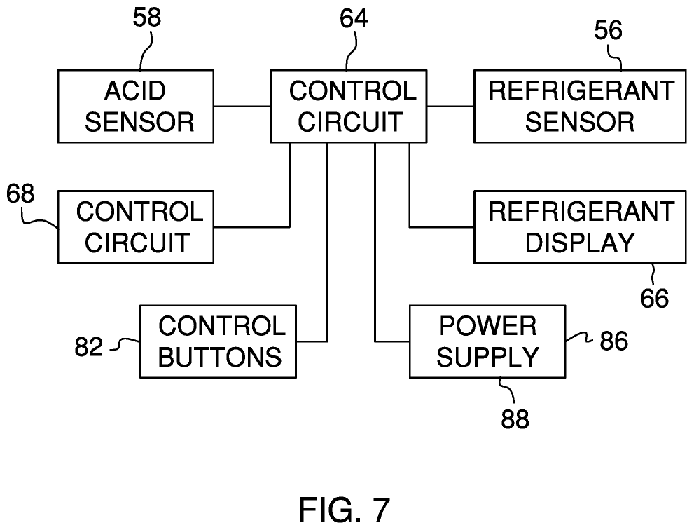

With reference now to the drawings, and in particular to FIGS. 1 through 7 thereof, a new identification device embodying the principles and concepts of an embodiment of the disclosure and generally designated by the reference numeral 10 will be described.

As best illustrated in FIGS. 1 through 7, the refrigerant identification assembly 10 generally comprises a housing 12 that has a high pressure inlet 14, a low pressure inlet 16 and a purge outlet 18. The housing 12 has a front wall 20, a back wall 22 and a perimeter wall 24 extending therebetween, and the perimeter wall 24 has a bottom side 26, a top side 28, a first lateral side 30 and a second lateral side 32. The purge outlet 18 is positioned on the top side 28 of the perimeter wall 24 and each of the high pressure inlet 14 and the low pressure inlet 16 is positioned on the bottom side 26 of the perimeter wall 24. Additionally, each of the high pressure inlet 14 and the low pressure inlet 16 is aligned with a respective one of the first lateral side 30 or the second lateral side 32 of the perimeter wall 24. The back wall 22 has a well 34 extending inwardly therein and the well 34 has a first portion 36 extending between a pair of second portions 38. Each of the second portions 38 is oriented perpendicular to the first portion 36 and the second portions 38 are spaced apart from each other such that the well 34 has a U-shape with the second portions 38 directed toward the top side 28 of the perimeter wall 24.

A high pressure hose 40 is provided and the high pressure hose 40 is fluidly coupled to the high pressure inlet 14. The high pressure hose 40 can be fluidly coupled to a high pressure refrigerant line of a heating, ventilation, and air conditioning (HVAC) system 41 thereby facilitating a high pressure refrigerant to be delivered into the housing 12. The high pressure hose 40 has a distal end 42 with respect to the high pressure inlet 14 and a high pressure fitting 44 is fluidly coupled to the distal end 42 of the high pressure hose 40. The high pressure fitting 44 can be fluidly coupled to the high pressure refrigerant line 46. The high pressure fitting 44 may be a threaded coupling commonly employed on HVAC systems.

A low pressure hose 48 is provided and the low pressure hose 48 is fluidly coupled to the low pressure inlet 16. The low pressure hose 48 can be fluidly coupled to a low pressure refrigerant line 50 of the HVAC system 41 thereby facilitating a low pressure refrigerant to be delivered into the housing 12. The low pressure hose 48 has a distal end 52 with respect to the low pressure inlet 16 and a low pressure fitting 54 is fluidly coupled to the distal end of the low pressure hose 48. The low pressure fitting 54 can be fluidly coupled to the low pressure refrigerant line. Additionally, the low pressure fitting 54 may be a threaded coupling commonly employed on a low pressure circuit of HVAC systems.

A refrigerant sensor 56 is provided and the refrigerant sensor 56 is positioned within the housing 12. The refrigerant sensor 56 is in fluid communication with each of the high pressure inlet 14 and the low pressure inlet 16. In this way the refrigerant sensor 56 can sense the type of refrigerant used in the HVAC system 41 when the high pressure hose 40 and the low pressure hose 48 are fluidly coupled to the HVAC system 41. The refrigerant sensor 56 may be an electronic Freon sensor or the like that can distinguish between R-22 refrigerant and R-410A refrigerant.

An acid sensor 58 is provided and the acid sensor 58 is positioned within the housing 12. The acid sensor 58 is in fluid communication with each of the high pressure inlet 14 and the low pressure inlet 16 to sense acid in the refrigerant used in the air conditioning system. The acid sensor 58 may be an electronic chemical sensor or the like that can detect acids in the refrigerant lines that would indicate a potential mechanical failure of a compressor in the HVAC system 41.

A purge valve 60 is positioned within the housing 12 and the purge valve 60 is in fluid communication between the high pressure inlet 14, the low pressure inlet 16 and the purge outlet 18. The purge valve 60 is positionable in an open condition to release the refrigerant through the purge outlet 18. Conversely, the purge valve 60 is biased into a closed condition to inhibit the refrigerant from being released through the purge outlet 18. A purge button 62 is movably coupled to the housing 12 and the purge button 62 can be depressed by a user. The purge button 62 is in mechanical communication with the purge valve 60 and the purge valve 60 is positioned in the open condition when the purge button 62 is depressed. Additionally, the purge valve 60 is biased into the closed condition when the purge button 62 is not depressed.

A control circuit 64 is provided and the control circuit 64 is positioned within the housing 12. The control circuit 64 is electrically coupled to each of the refrigerant sensor 56 and the acid sensor 58. A refrigerant display 66 is coupled to the housing 12 such that the refrigerant display 66 is visible to the user. The refrigerant display 66 is electrically coupled to the control circuit 64 and the refrigerant display 66 displaying indicia 70 comprising letters and numbers for identifying the type of refrigerant sensed by the refrigerant sensor 56. In this way the refrigerant display 66 can communicate the type of refrigerant to the user.

An acid display 68 is coupled to the housing 12 such that the acid display 68 is visible to the user. The acid display 68 is electrically coupled to the control circuit 64 and the acid display 68 displays indicia 70 comprising letters and numbers for identifying the type and quantity of acid sensed by the acid sensor 58. In this way the acid display 68 communicates the type and quantity of acid to the user. Each of the refrigerant display 66 and the acid display 68 may comprise an LED or other type of electronic display.

A stand 72 is pivotally coupled to the housing 12 and the stand 72 is positionable in a deployed position for standing the housing 12 on a support surface 74. The support surface 74 may be a heat pump of the HVAC system 41 or the like. The stand 72 is positionable in a stored position. The stand 72 comprises a first member 76 extending between a pair of second members 78, and each of the second members 78 has a distal end 80 with respect to the first member 76.

Each of the second members 78 is oriented perpendicular to the first member 76 and the distal end 80 of each of the second members 78 is pivotally coupled to the back wall 22 of the housing 12. Each of the second members 78 is positioned in a respective one of the second portions 38 of the well 34 in the back wall 22 of the housing 12 when the stand 72 is positioned in the stored position. The first member 76 is positioned in the first portion 36 of the well 34 in the back wall 22 of the housing 12 when the stand 72 is positioned in the stored position. Moreover, each of the second members 78 angles away from the back wall 22 of the housing 12 to rest on the support surface 74.

A plurality of control buttons 82 is each movably coupled to the housing 12. Each of the control buttons 82 controls respective operational parameters of the refrigerant sensor 56 and the acid sensor 58. Each of the control buttons 82 is electrically coupled to the control circuit 64. The plurality of control buttons 82 may include, but not be limited to, a power button, a sensitivity increase button, a sensitivity decrease button and a menu button.

A battery cover 84 is removably positioned over a battery space 85 that is recessed into the back wall 22 of the housing 12. A power supply 86 is provided and the power supply 86 is removably coupled to the housing 12. The power supply 86 is electrically coupled to the control circuit 64 and the power supply 86 comprises at least one battery 88. Moreover, the battery 88 is positioned in the battery space 85 and is covered by the battery cover 84.

In use, each of the high pressure fitting 44 and the low pressure fitting 54 are fluidly coupled to respective high pressure refrigerant line and low pressure refrigerant line on the HVAC system 41. In this way the refrigerant sensor 56 and the acid sensor 58 are exposed to the refrigerant used in the HVAC system 41. Thus, the type of refrigerant is identified for the user as well as the acid content of the refrigerant. In this way the type of refrigerant can be precisely identified compared to the traditional method of interpreting a pressure gauge reading for extrapolating the most likely type of refrigerant in the HVAC system 41. Moreover, the user can quickly perform a diagnosis of potential compressor failure with the acid levels detected in the refrigerant. In this way non-invasive tests can be performed on the HVAC system 41 prior to performing service work on the HVAC system 41. Additionally, the purge outlet 18 is fluidly coupled to a desired purge line to facilitate refrigerant to be purged from the housing 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A refrigerant identification assembly for identifying the type of refrigerant used in an air conditioning system, said assembly comprising:
a housing having a high pressure inlet, a low pressure inlet and a purge outlet;
a high pressure hose being fluidly coupled to said high pressure inlet wherein said high pressure hose is configured to be fluidly coupled to a high pressure refrigerant line of an HVAC system thereby facilitating a high pressure refrigerant to be delivered into said housing;
a low pressure hose being fluidly coupled to said low pressure inlet wherein said low pressure hose is configured to be fluidly coupled to a low pressure refrigerant line of an HVAC system thereby facilitating a low pressure refrigerant to be delivered into said housing;
a refrigerant sensor being positioned within said housing, said refrigerant sensor being in fluid communication with each of said high pressure inlet and said low pressure inlet wherein said refrigerant sensor is configured to sense the type of refrigerant used in the air conditioning system;
an acid sensor being positioned within said housing, said acid sensor being in fluid communication with each of said high pressure inlet and said low pressure inlet wherein said acid sensor is configured to sense acid in the refrigerant used in the air conditioning system;
a refrigerant display being coupled to said housing wherein said refrigerant display is configured to be visible to the user, said refrigerant display being in communication with said refrigerant sensor wherein said refrigerant display is configured to communicate the type of refrigerant to the user; and
an acid display being coupled to said housing wherein said acid display is configured to be visible to the user, said acid display being in communication with said acid sensor wherein said acid display is configured to communicate the type and quantity of acid to the user.

2. The assembly according to claim 1, wherein said housing has a front wall, a back wall and a perimeter wall extending therebetween, said perimeter wall having a bottom side, a top side, a first lateral side and a second lateral side, said purge outlet being positioned on said top side of said perimeter wall, each of said high pressure inlet and said low pressure inlet being positioned on said bottom side of said perimeter wall, each of said high pressure inlet and said low pressure inlet being aligned with a respective one of said first lateral side or said second lateral side of said perimeter wall.

3. The assembly according to claim 2, wherein said back wall having a well extending inwardly therein, said well having a first portion extending between a pair of second portions, each of said second portions being oriented perpendicular to said first portion, said second portions being spaced apart from each other such that said well has a U-shape having said second portions being directed toward said top side of said perimeter wall.

4. The assembly according to claim 1, wherein:
said high pressure hose has a distal end with respect to said high pressure inlet; and
said assembly includes a high pressure fitting being fluidly coupled to said distal end of said high pressure hose wherein said high pressure fitting is configured to be fluidly coupled to the high pressure refrigerant line.

5. The assembly according to claim 1, wherein:
said low pressure hose has a distal end with respect to said low pressure inlet; and
said assembly includes a low pressure fitting being fluidly coupled to said distal end of said low pressure hose wherein said low pressure fitting is configured to be fluidly coupled to the low pressure refrigerant line.

6. The assembly according to claim 1, further comprising a purge valve being positioned within said housing, said purge valve being in fluid communication between said high pressure inlet, said low pressure inlet and said purge outlet, said purge valve being positionable in an open condition wherein said purge valve is configured to release the refrigerant through said purge outlet, said purge valve being biased into a closed condition wherein said purge valve is configured to inhibit the refrigerant from being released through said purge outlet.

7. The assembly according to claim 6, further comprising a purge button being movably coupled to said housing wherein said purge button is configured to be depressed by a user, said purge button being in mechanical communication with said purge valve, said purge valve being positioned in said open condition when said purge button is depressed, said purge valve being biased into said closed condition when said purge button is not depressed.

8. The assembly according to claim 1, further comprising a control circuit being positioned within said housing, said control circuit being electrically coupled to said refrigerant sensor, said control circuit being electrically coupled to said acid sensor.

9. The assembly according to claim 8, wherein said refrigerant display is electrically coupled to said control circuit, said refrigerant display refrigerant displaying indicia comprising letters and numbers for identifying the type of refrigerant sensed by said refrigerant sensor.

10. The assembly according to claim 8, wherein said acid display is electrically coupled to said control circuit, said acid display displaying indicia comprising letters and numbers for identifying the type and quantity of acid sensed by said acid sensor.

11. The assembly according to claim 2, further comprising a stand being pivotally coupled to said housing, said stand being positionable in a deployed position having said stand angling away from said housing for standing said housing on a support surface, said stand being positionable in a stored position.

12. The assembly according to claim 11, wherein said stand comprises a first member extending between a pair of second members, each of said second members having a distal end with respect to said first member, each of said second members being oriented perpendicular to said first member, said distal end of each of said second members being pivotally coupled to said back wall of said housing.

13. The assembly according to claim 12, wherein each of said second members is positioned in a respective one of said second portions of said well in said back wall of said housing when said stand is positioned in said stored position, said first member being positioned in said first portion of said well in said back wall of said housing when said stand is positioned in said stored position, each of said second members angling away from said back wall of said housing wherein said first member is configured to rest on the support surface.

14. The assembly according to claim 8, further comprising a plurality of control buttons, each of said control buttons being movably coupled to said housing, each of said control buttons controlling respective operational parameters of said refrigerant sensor and said acid sensor, each of said control buttons being electrically coupled to said control circuit.

15. A refrigerant identification assembly for identifying the type of refrigerant used in an air conditioning system, said assembly comprising:
a housing having a high pressure inlet, a low pressure inlet and a purge outlet, said housing having a front wall, a back wall and a perimeter wall extending therebetween, said perimeter wall having a bottom side, a top side, a first lateral side and a second lateral side, said purge outlet being positioned on said top side of said perimeter wall, each of said high pressure inlet and said low pressure inlet being positioned on said bottom side of said perimeter wall, each of said high pressure inlet and said low pressure inlet being aligned with a respective one of said first lateral side or said second lateral side of said perimeter wall, said back wall having a well extending inwardly therein, said well having a first portion extending between a pair of second portions, each of said second portions being oriented perpendicular to said first portion, said second portions being spaced apart from each other such that said well has a U-shape having said second portions being directed toward said top side of said perimeter wall;
a high pressure hose being fluidly coupled to said high pressure inlet wherein said high pressure hose is configured to be fluidly coupled to a high pressure refrigerant line of an HVAC system thereby facilitating a high pressure refrigerant to be delivered into said housing, said high pressure hose having a distal end with respect to said high pressure inlet;
a high pressure fitting being fluidly coupled to said distal end of said high pressure hose wherein said high pressure fitting is configured to be fluidly coupled to the high pressure refrigerant line;
a low pressure hose being fluidly coupled to said low pressure inlet wherein said low pressure hose is configured to be fluidly coupled to a low pressure refrigerant line of an HVAC system thereby facilitating a low pressure refrigerant to be delivered into said housing, said low pressure hose having a distal end with respect to said low pressure inlet;
a low pressure fitting being fluidly coupled to said distal end of said low pressure hose wherein said low pressure fitting is configured to be fluidly coupled to the low pressure refrigerant line;
a refrigerant sensor being positioned within said housing, said refrigerant sensor being in fluid communication with each of said high pressure inlet and said low pressure inlet wherein said refrigerant sensor is configured to sense the type of refrigerant used in the air conditioning system;
an acid sensor being positioned within said housing, said acid sensor being in fluid communication with each of said high pressure inlet and said low pressure inlet wherein said acid sensor is configured to sense acid in the refrigerant used in the air conditioning system;
a purge valve being positioned within said housing, said purge valve being in fluid communication between said high pressure inlet, said low pressure inlet and said purge outlet, said purge valve being positionable in an open condition wherein said purge valve is configured to release the refrigerant through said purge outlet, said purge valve being biased into a closed condition wherein said purge valve is configured to inhibit the refrigerant from being released through said purge outlet;
a purge button being movably coupled to said housing wherein said purge button is configured to be depressed by a user, said purge button being in mechanical communication with said purge valve, said purge valve being positioned in said open condition when said purge button is depressed, said purge valve being biased into said closed condition when said purge button is not depressed;
a control circuit being positioned within said housing, said control circuit being electrically coupled to said refrigerant sensor, said control circuit being electrically coupled to said acid sensor;
a refrigerant display being coupled to said housing wherein said refrigerant display is configured to be visible to the user, said refrigerant display being electrically coupled to said control circuit, said refrigerant display refrigerant displaying indicia comprising letters and numbers for identifying the type of refrigerant sensed by said refrigerant sensor wherein said refrigerant display is configured to communicate the type of refrigerant to the user;
an acid display being coupled to said housing wherein said acid display is configured to be visible to the user, said acid display being electrically coupled to said control circuit, said acid display displaying indicia comprising letters and numbers for identifying the type and quantity of acid sensed by said acid sensor wherein said acid display is configured to communicate the type and quantity of acid to the user;
a stand being pivotally coupled to said housing, said stand being positionable in a deployed position having said stand angling away from said housing for standing said housing on a support surface, said stand being positionable in a stored position, said stand comprising a first member extending between a pair of second members, each of said second members having a distal end with respect to said first member, each of said second members being oriented perpendicular to said first member, said distal end of each of said second members being pivotally coupled to said back wall of said housing, each of said second members being positioned in a respective one of said second portions of said well in said back wall of said housing when said stand is positioned in said stored position, said first member being positioned in said first portion of said well in said back wall of said housing when said stand is positioned in said stored position, each of said second members angling away from said back wall of said housing wherein said first member is configured to rest on the support surface;
a plurality of control buttons, each of said control buttons being movably coupled to said housing, each of said control buttons controlling respective operational parameters of said refrigerant sensor and said acid sensor, each of said control buttons being electrically coupled to said control circuit; and
a power supply being removably coupled to said housing, said power supply being electrically coupled to said control circuit, said power supply comprising at least one battery.
